(19) United States
(12) Patent Application Publication
(10) Pub. No.: US 2009/0030421 A1
(43) Pub. Date: Jan. 29, 2009

(54) Implant engagement method and device

(75) Inventors: J. Riley Hawkins, Cumberland, RI (US); Paul Birkmeyer, Marshfield, MA (US); Brett R. Zarda, Providence, RI (US); Katherine Torres, Westport, MA (US); Katherine Herard, South Boston, MA (US)

Correspondence Address: MAGINOT, MOORE & BECK, LLP;CHASE TOWER, 111 MONUMENT CIRCLE, SUITE 3250, INDIANAPOLIS, IN, 46204, US

(73) Assignee: DePuy Spine, Inc., Raynham, MA (US)

(21) Appl. No.: 11/880,525

(22) Filed: Jul. 23, 2007

(51) Int. Cl. A61F 2/46

(52) U.S. Cl. 606/99

(57) ABSTRACT

A method and system for engaging an implant with a bone is disclosed. In one method incorporating principles of the invention, a bone is engaged with an implant by placing a first surface of an implant adjacent to a first bone portion, contacting the first bone portion with at least one first engagement member extending from the first surface, controlling an agitator to agitate the first surface of the implant and the at least one first engagement member, generating at least one first surface feature in the first bone portion with the agitated at least one first engagement member, stilling the first surface implant and the at least one first engagement member and settling the stilled at least one first engagement member into engagement with the at least one first surface feature.

### FIELD OF THE INVENTION

This invention relates to surgical methods and devices and, more particularly, to methods and devices used to facilitate engagement of devices with a bone.

### BACKGROUND

The spine is made of bony structures called vertebral bodies that are separated by soft tissue structures called intervertebral discs. The intervertebral disc is commonly referred to as a spinal disc. The spinal disc primarily serves as a mechanical cushion between the vertebral bones, permitting controlled motions between vertebral segments of the axial skeleton. The disc acts as a synchondral joint and allows some amount of flexion, extension, lateral bending, and axial rotation.

The normal disc is a mixed avascular structure including two vertebral end plates, annulus fibrosis and nucleus pulposus. The end plates are composed of thin cartilage overlying a layer of hard, cortical bone that attaches to the spongy cancellous bone of the adjacent vertebral body.

The discs are subjected to a variety of loads as the posture of an individual changes. Even when the effects of gravity are removed, however, the soft tissue connected to the spine generates a compressive force along the spine. Thus, even when the human body is supine, the compressive load on the third lumbar disc is on the order of 300 Newtons (N).

The spinal disc may be displaced or damaged due to trauma or a disease process. A disc herniation occurs when the annulus fibers are weakened or torn and the inner material of the nucleus becomes permanently bulged, distended, or extruded out of its normal, internal annular confines. The mass of a herniated or “slipped” nucleus tissue can compress a spinal nerve, resulting in leg pain, loss of muscle strength and control or even paralysis. Alternatively, with discal degeneration, the nucleus loses its water binding ability and dehydrates with subsequent loss in disc height. Consequently, the volume of the nucleus decreases, causing the annulus to buckle in areas where the laminated plies are loosely bonded. As these overlapping plies of the annulus buckle and separate, either circumferential or radial annular tears may occur, potentially resulting in persistent and disabling back pain. Adjacent, ancillary facet joints will also be forced into an overriding position, which may cause additional back pain.

Recently, efforts have been directed to replacing defective spinal column components including intervertebral discs. Some replacement components use a solid core of elastomeric material, such as polyolefin, to act as a compressible core between two metal endplates. The metal endplates are typically engaged to the adjacent intervertebral bodies by spikes which extend from the outer surface of the metal endplate. Engagement of the spikes is achieved by impacting the endplate so as to drive the spikes into the bony structure of the adjacent intervertebral body. Properly seating the endplate in this fashion, however, presents various problems.

As an initial matter, access to the spinal area is generally achieved either through an anterior, posterior or lateral incision that is directly aligned with the area of the spine to be operated upon. Embedment of the endplate, however, requires a force to be applied orthogonal to the incision path. Thus, the impacting tool will normally contact the end plate at some angle off of the longitudinal axis of the spinal column. Therefore, the spikes on the endplate which are closest to the impacting tool may be fully engaged while those on the opposite side of the endplate are only partially engaged.

Moreover, because the impact is provided at an angle, much of the force of the impact is wasted. Furthermore, the wasted impact tends to force the metal endplate away from the incision point and out of alignment with the spinal column. This problem is exacerbated by a recent trend toward minimally invasive surgery. Specifically, as the incision providing access to the spinal column decreases in size, the angular constraints on the tools and instruments used in the surgery become more restricted.

A need exists for a system and method which allows endplates of an implant to be more easily attached to bone. A further need exists for a system and method which can be used in a minimally invasive surgery. It would be advantageous if the system and method could be used with a variety of geometric relationships between the location of an incision and the location of the implant.

### SUMMARY

A method and system for engaging an implant with a bone is disclosed. In one method incorporating principles of the invention, a bone is engaged with an implant by placing a first surface of an implant adjacent to a first bone portion, contacting the first bone portion with at least one first engagement member extending from the first surface, controlling an agitator to agitate the first surface of the implant and the at least one first engagement member, generating at least one first surface feature in the first bone portion with the agitated at least one first engagement member, stilling the first surface implant and the at least one first engagement member and settling the stilled at least one first engagement member into engagement with the at least one first surface feature.

In accordance with another embodiment, an implant positioning tool includes a housing, an agitator located within the housing for providing a recurring pattern of movement and a shaft extending out of the housing and having a first end portion operably connected to the agitator and a second end portion configured to operably couple with an implant such that the recurring pattern of movement of the agitator causes the implant to move in a recurring pattern corresponding to the recurring pattern of movement of the agitator.

The above-described features and advantages, as well as others, will become more readily apparent to those of ordinary skill in the art by reference to the following detailed description and accompanying drawings.

### BRIEF DESCRIPTION OF THE DRAWINGS

FIG. 1 shows a cross-sectional view of an insertion instrument incorporating principles of the present invention;

FIG. 2 shows a perspective view of one embodiment of a gripper that can be used with the insertion instrument of FIG. 1 in accordance with principles of the present invention;

FIG. 3 shows a perspective view of one embodiment of an artificial intervertebral disc that may be gripped using the gripper of FIG. 2;

FIG. 4 shows a cross-sectional view of the insertion instrument of FIG. 1 with the trigger mechanism in a released position;

FIG. 5 shows a cross-sectional view of the insertion instrument of FIG. 1 with the trigger mechanism in a compressed position;

FIG. 6 shows a cross-sectional view of the insertion instrument of FIG. 1 with the trigger mechanism in a compressed position and the gripper of FIG. 2 attached to the internal shaft of the insertion instrument;

FIG. 7 shows a partial perspective view of the insertion instrument of FIG. 1 and the gripper of FIG. 2 snugly gripping the artificial intervertebral disc of FIG. 3;

FIG. 8 shows a cross-sectional view of the insertion instrument of FIG. 1 with the trigger mechanism in a released position and the gripper of FIG. 2 attached to the internal shaft of the insertion instrument such that the finger pairs or the gripper are forced toward each other;

FIG. 9 shows a partial plan view of an intervertebral disc space created between two vertebrae which have been distracted in accordance with principles of the present invention;

FIG. 10 shows a partial plan view of the intervertebral disc space created between the two vertebrae of FIG. 9 with the insertion instrument of FIG. 1 and the gripper of FIG. 2 used to securely grip the artificial disc of FIG. 3 and to position the artificial disc of FIG. 3 within the intervertebral disc space in accordance with principles of the present invention;

FIG. 11 shows a partial plan view of the intervertebral disc space and artificial disc of FIG. 10 after at least some of the distraction force on the vertebrae has been reduced;

FIG. 12 is a schematic partial plan view of the artificial disc of FIG. 3 showing the movement of an engagement member when a movement vector of the artificial disc parallel to the axis of the insertion instrument is about ½ of the length of the footprint of the engagement member on the endplate of the artificial disc;

FIG. 13 is a schematic partial plan view showing the area of bone that is swept by the movement of the engagement member of FIG. 12;

FIG. 14 is a schematic partial plan view of the artificial disc of FIG. 3 showing the movement of an engagement member when a movement vector of the artificial disc parallel to the axis of the insertion instrument is significantly less than ½ of the length of the footprint of the engagement member on the endplate of the artificial disc;

FIG. 15 is a schematic partial plan view showing the area of bone that is swept by the movement of the engagement member of FIG. 14; and

FIG. 16 shows a partial plan view of the intervertebral disc space and artificial disc of FIG. 10 after the artificial disc has been embedded into the adjacent vertebrae and released.

### DETAILED DESCRIPTION

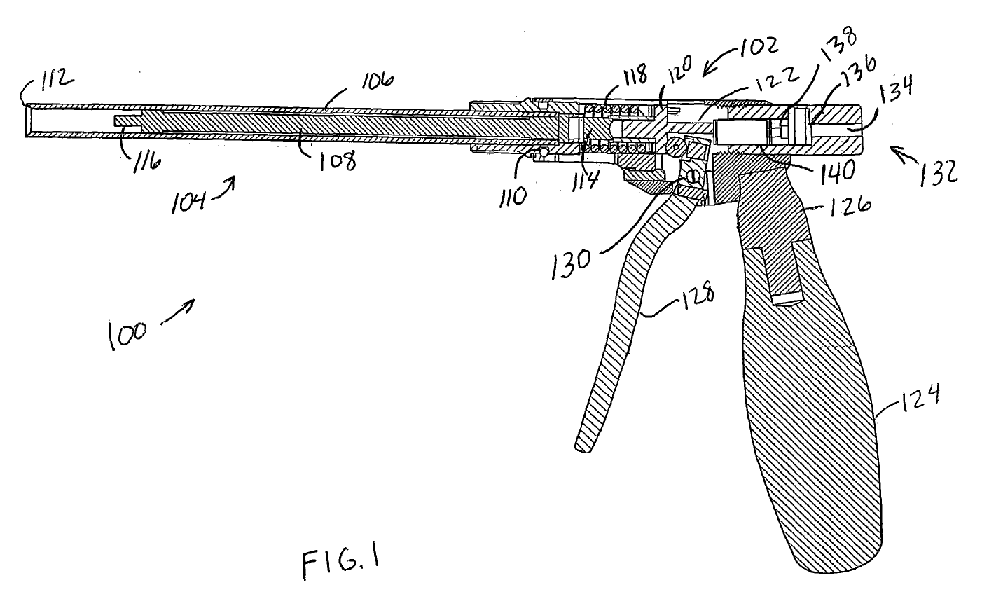

FIG. 1 depicts a side cross-sectional view of an insertion instrument 100. The insertion instrument 100 includes a body housing 102 and a sheath portion 104. The sheath portion 104 includes an outer sleeve 106 which encloses an inner shaft 108 and which is retained by a retaining pin 110. The outer sleeve 106 includes a tapered end portion 112. The inner shaft 108 includes a female threaded end 114 and a male threaded end 116.

An internal compression spring 118 is fastened to the sheath portion 104 and held in place by a spring retaining screw 120 which is threadedly engaged with the female threaded end 114 of the inner shaft 108. The spring retaining screw 120 includes a drive shaft 122 which extends along the axis of the insertion instrument 100. Once the sheath portion 104 is assembled, it is inserted into the body housing 102 and retained within the body housing 102 with the retaining pin 110.

The body housing 102 includes a handle 124, a handle transition 126, a trigger mechanism 128, and pivot pin 130. The trigger mechanism 128 can be any type of trigger mechanism known in the art. The trigger mechanism 128 of FIG. 1 pivots about the pivot pin 130 in the body housing 102.

The body housing 102 is configured to threadingly receive an agitator component 132 which includes a port 134 for the insertion of a power source. The power source may be a power cord or a battery pack. Energy from the power source is used to drive a transducer 136. The transducer 136 is in operable contact with a driver 138 and armature 140. When the agitator component 132 is threaded into the body housing 102 and the trigger mechanism 128 is in the position shown in FIG. 1, the drive shaft 122 is operably received within the armature 140.

The transducer 136 in this embodiment includes a piezoelectric driver which contains Thunder Technology, which is a high deformation Piezo electrical actuator, (described and illustrated in U.S. Pat. No. 5,632,841, U.S. Pat. No. 5,639,850 and U.S. Pat. No. 6,030,480, the disclosures of which are incorporated herein by reference). The transducer provides operating frequencies of between 40 kHz and 65 kHz, although other frequencies may be used.

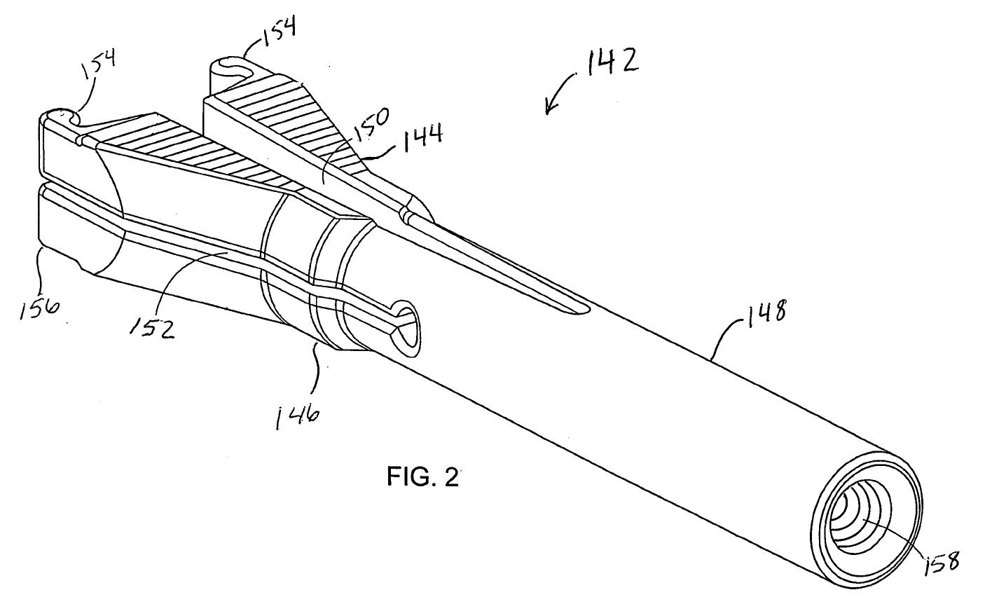

FIG. 2 shows a gripper 142 which includes a coupling portion 144, a throat portion 146 and a shaft 148 in an unstressed condition. The coupling portion 144 includes a slit 150 and a slit 152 which extend through the coupling portion 144 and the throat portion 146 into the shaft 148. The slits 150 and 152 define two opposing pairs of fingers 154 and 156 in the coupling portion 144 (only one finger of finger pair 156 is shown in FIG. 2). The throat portion 146 tapers from a larger diameter at the coupling portion 144 to a smaller diameter at the shaft 148. The shaft 148 includes a threaded inner bore 158 which is configured to be engaged with the male threaded end 116 of the inner shaft 108.

The coupling portion 144 of the gripper 142 is configured to mate with an artificial disc such as the artificial disc 160 shown in FIG. 3. The artificial disc 160 includes two endplates 162 and 164 which are separated by a core 166. Each of the two endplates 162 and 164 include a number of engagement members 168. In the embodiment of FIG. 3, the engagement members 168 are generally in the shape of a cone, with the apex 170 of the engagement members 168 spaced apart from the respective endplate 162 or 164. In alternative embodiments, the engagement members may be pyramidal, conical, or another shape. Preferably, the portions of the engagement members farthest away from the endplates, such as the apex of the engagement members 168, are relatively sharp.

The endplates 162 and 164 further include four notches 172, 174, 176 and 178 and four notches including the notch 180 and three notches not shown) that are symmetrical and spaced apart from the notches 172, 174, 176 and 178 to form four notch pairs. By way of example, the notch 180 which is shown in FIG. 3 in shadow form, is the symmetrical to and spaced apart notch for the notch 172. Thus, the notch 172 and the notch 180 area notch pair.

The eight notches, 172, 174, 176, 178, 180, and the three notches not shown, are sized and shaped to snugly mate with the fingers in the finger pairs 154 and 156. Additionally, the notches 172 and 176 define a ledge 182 which is sized for engagement with the width of the slit 152. Moreover, the distance between each of the notches in the notch pairs is substantially the same as the distance between the opposing fingers of the finger pairs 154 and 156.

Operation of the insertion instrument 100 begins with the insertion instrument 100 in the condition of FIG. 4. In FIG. 4, the trigger mechanism 128 is not depressed. Accordingly, the trigger mechanism is maintained in the position of FIG. 4 by the internal compression spring 118, which is configured to bias the inner shaft 108 to the rear of the insertion instrument 100 which, in FIG. 4, is to the right. Specifically, the internal compression spring 118 forces the spring retaining screw 120 against the trigger mechanism 128.

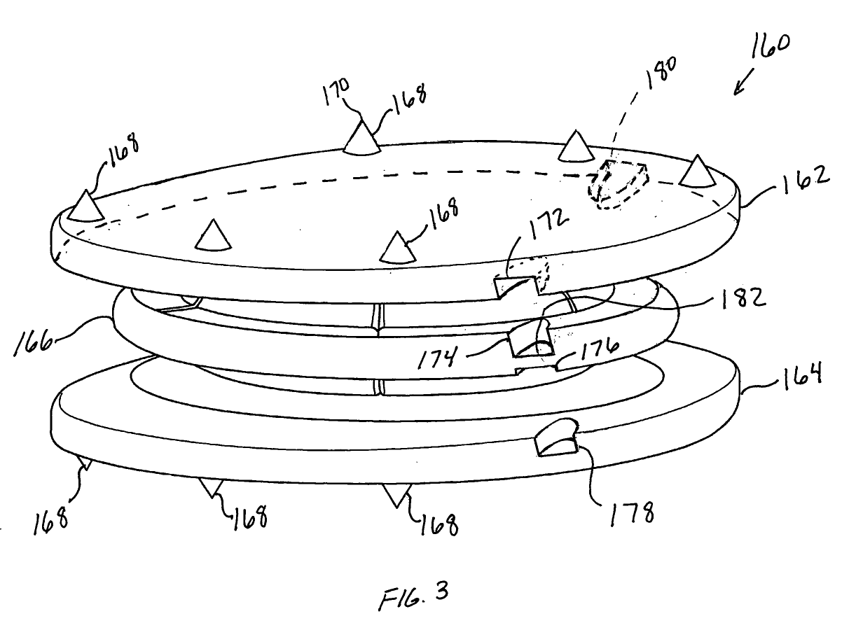

Next, the operator applies a force to the trigger mechanism 128 in the direction of the arrow 184. As the force applied to the trigger mechanism 128 increases above the force provided by the internal compression spring 118, the trigger mechanism 128 pivots about the pivot pin 130 forcing the spring retaining screw 120 in the direction of the arrow 186. As the spring retaining screw 120 moves in the direction of the arrow 186, the internal compression spring 118 is compressed and the inner shaft 108 is forced in the direction of the arrow 186 to the position shown in FIG. 5. If desired, a locking mechanism may be provided to maintain the trigger mechanism 128 in the compressed position of FIG. 5.

When the trigger mechanism 128 is fully compressed, the shaft 148 of the gripper 142 is inserted into the outer sleeve 106 of the insertion instrument 100. The threaded inner bore 158 of the gripper 142 is then positioned about the male threaded end 116 of the inner shaft 108 and threaded onto the male threaded end 116 to the position shown in FIG. 6. In the position of FIG. 6, the trigger mechanism 128 is fully compressed and the threaded inner bore 158 of the gripper 142 is fully engaged with the male threaded end 116 of the inner shaft 108. Additionally, the throat portion 146 of the gripper 142 is located adjacent to the tapered end portion 112 of the outer sleeve 106 and the slits 150 and 152 are in an uncompressed state.

Next, the gripper 142 is engaged to the artificial disc 160. This is accomplished by aligning the finger pair 154 with the notch pair 172 and 180 and the notch pair 182 and the symmetrical and spaced apart notch (not shown) for the notch 182. Additionally, the finger pair 156 is aligned with the notch pair 176 and the symmetrical and spaced apart notch (not shown) for the notch 176, and the notch pair 178 and the symmetrical and spaced apart notch (not shown) for the notch 178.

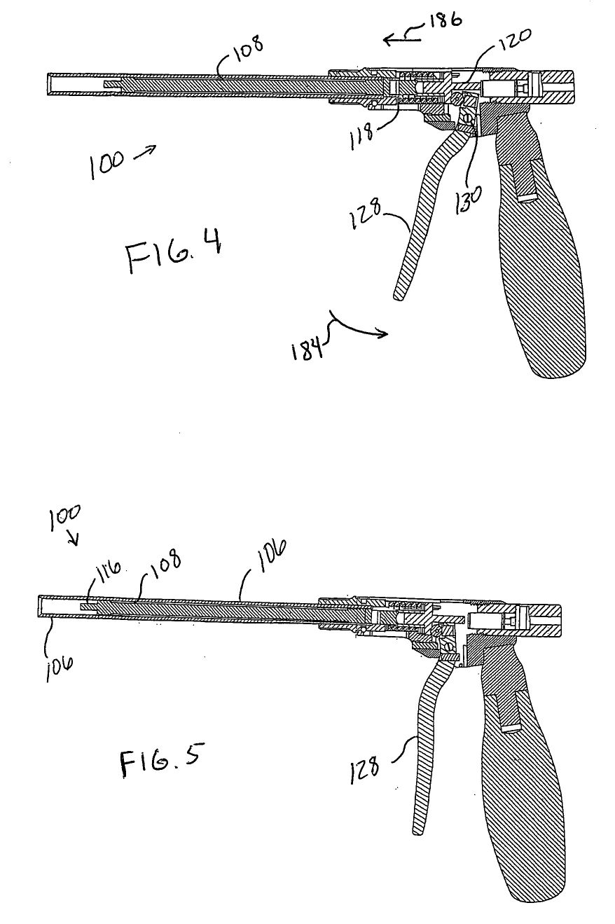

The gripper 142 is then pushed against the artificial disc 160. This force causes the fingers in the finger pairs 154 and 156 to be forced apart as the slit 150 widens. Additionally, in this embodiment, the finger pairs 154 and 156 are forced apart as the slit 152 widens. As the finger pairs 154 and 156 encounter the eight notches, 172, 174, 176, 178, 180 and the three notches not shown, the gripper 142 moves toward its non-stressed condition with the slit 150 narrowing and the finger pairs 154 and 156 moving into the eight notches, 172, 174, 176, 178, 180 and the three notches not shown. Thus, the artificial disc 160 is firmly gripped by the gripper 142 as shown in FIG. 7.

The operator now releases the trigger mechanism 128. As the force applied to the spring retaining screw 120 by the trigger mechanism 128 decreases below the force provided by the internal compression spring 118 on the spring retaining screw 120, the spring retaining screw 120 is forced in the direction of the arrow 188 as the internal compression spring 118 is decompressed and the inner shaft 108 is forced in the direction of the arrow 188. As the spring retaining screw 120 moves in the direction of the arrow 188, the drive shaft 122 is positioned within the armature 140 and the trigger mechanism 128 pivots about the pivot pin 130 in the direction indicated by the arrow 190.

Movement of the inner shaft 108 in the direction of the arrow 188 also forces the gripper 142 to be moved further into the outer sleeve 106. Specifically, the tapered end portion 112 acts upon the throat portion 146 of the gripper 142 thereby forcing the slit 150 and the slit 152 toward a narrower configuration. Accordingly, the finger pairs 154 and 156 are forced in a direction further into the eight notches, 172, 174, 176, 178, 180 and the three notches not shown and the finger pairs 154 and 156 are forced toward the ledge 182.

By way of example, FIG. 8 depicts the insertion instrument 100 with the trigger mechanism 128 in a non-compressed state and with the gripper 142 pulled further into the outer sleeve 106 than in the FIG. 6. Thus, the slit 152 is narrowed such that the finger pairs 154 and 156 are placed into contact with each other. Of course, when the artificial disc 160 is gripped by the gripper 142, the ledge 182 maintains the finger pairs 154 and 156 spaced apart from each other.

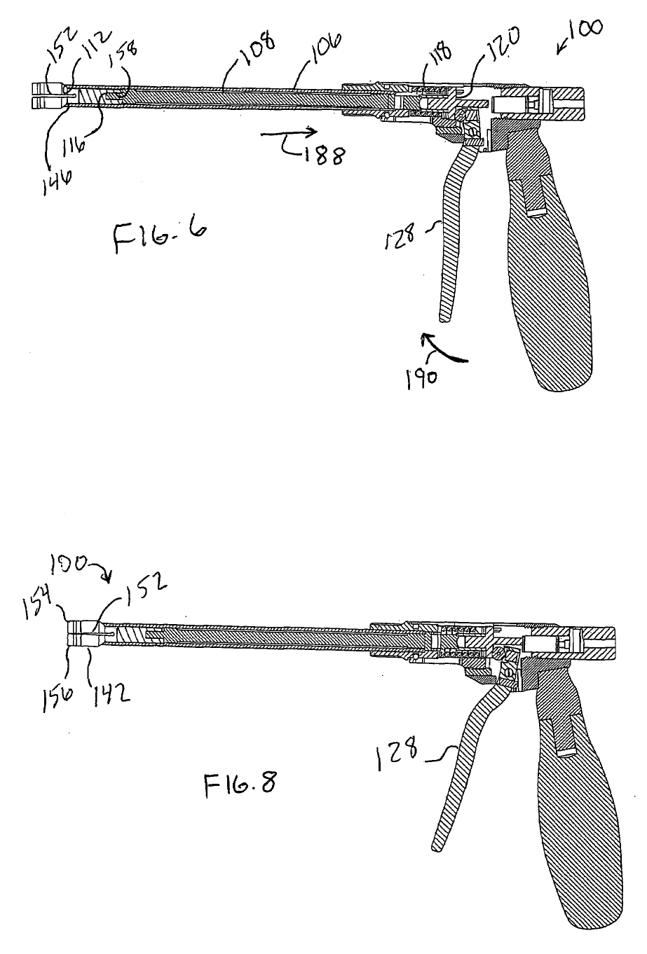

In this condition, the artificial disc 160 is securely gripped by the gripper 142. The insertion instrument 100 is then used to implant the artificial disc 160. In one method, the vertebrae 200 and 202 adjacent to an intervertebral disc to be replaced are distracted using a distractor (not shown) and the natural intervertebral disc is removed as shown in FIG. 9. The insertion instrument 100 is then used to position the artificial disc 160 in the intervertebral space between the vertebrae 200 and 202 as shown in FIG. 10. If desired, placement of the artificial disc 160 within the intervertebral space may be assisted by the use of guides. The guides may be integral with the distractor or separate components.

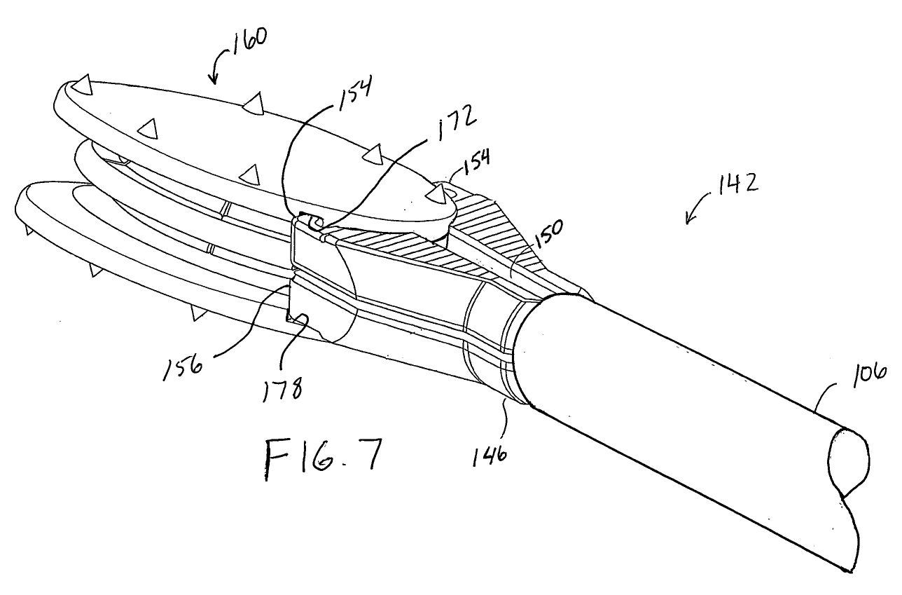

Once the artificial disc 160 is at the desired location, the force exerted on the vertebrae 200 and 202 by the distractor (not shown) is reduced. This allows the soft tissue connected to the spine to force the vertebrae 200 and 202 toward each other until the vertebrae 200 and 202 are partially embedded onto the artificial disc 160 as shown in FIG. 11. The force exerted by the soft tissue on the spine is not, however, sufficient to fully embed the vertebrae 200 and 202 onto the artificial disc 160.

With the artificial disc 160 securely gripped by the gripper 142 and partially embedded into the adjacent vertebrae 200 and 202, the agitator component 132 is activated. In this embodiment, the agitator component 132 generates a reciprocating movement of the drive shaft 122 along the axis of the insertion instrument 100 resulting in a repeated pattern of movement in the directions indicated by the arrows 204 and 206 in FIG. 11. Specifically, the movement of the drive shaft 122 is transferred to the inner shaft 108 through the female threaded end 114 of the inner shaft 108. The inner shaft 108 in turn causes the gripper 142 to move in the repeated pattern of movement in the directions indicated by the arrows 204 and 206. Therefore, because the artificial disc 160 is securely gripped by the gripper 142, the artificial disc 160 also moves in the same pattern generated by the agitator component 132.

The resultant movement of the engagement members 168 on the artificial disc 160 is depicted in FIG. 12. As the agitator component 132 causes movement in the direction of the arrow 204, the engagement member 168 moves from its original position to the position indicated by the engagement member 168′ which is offset from the original position of the engagement member 168 by ½ of the length of the footprint of the engagement member 168 on the endplate 162. The footprint of the engagement member 168 on the endplate 162 along the axis of the insertion instrument is identified by the points “A” and “B” in FIG. 12.

As the agitator component 132 causes movement in the direction of the arrow 206, the engagement member 168 moves to the position indicated by the engagement member 168″ which is offset from the original position of the engagement member 168 by ½ of the length of the footprint of the engagement member 168 on the endplate 162 in a direction opposite to the offset of the engagement member 168′ from the position of the engagement member 168. Accordingly, the amplitude of the movement in the axis of the insertion instrument 100 is equal to the length of the footprint of the engagement member 168 on the endplate 162 parallel to the axis of the insertion instrument 100.

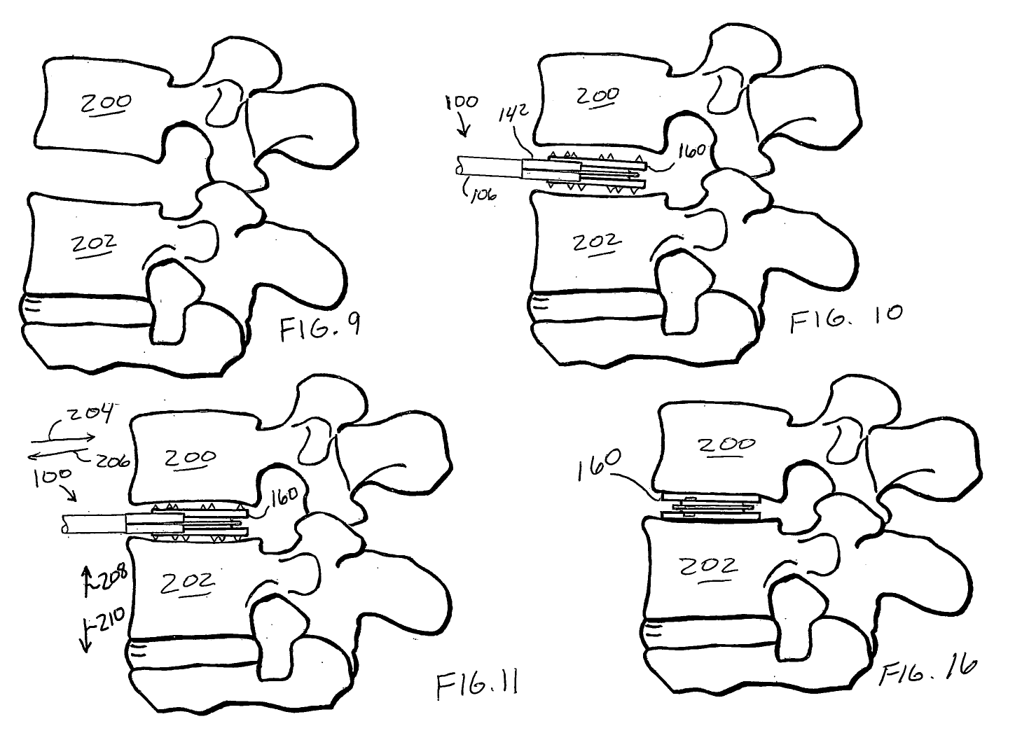

Thus, as shown in FIG. 13, the above described movement of the engagement member 168 causes the engagement member 168 to sweep an area “C” of the adjacent vertebra 200 or 202. The repeated movement of the engagement member 168 as pressure is applied to the vertebrae 200 and 202 by the soft tissue connected to the spine results in a scraping and/or compaction of the vertebra 200 or 202 at the contact point of the engagement member 168. Accordingly, an area in the bone corresponding to the area “C” is either scraped away or compacted leaving a surface feature in the vertebra 200 or 202 in which the engagement member 168 remains.

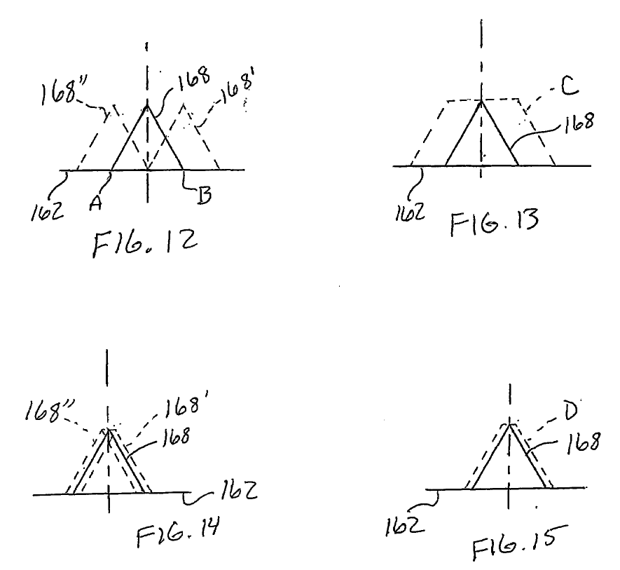

The final shape of the surface feature will depend upon the resiliency of the vertebral bone as well as the amplitude of the repeated movement and the size of the engagement member. Any resiliency of the vertebral bone will tend to reduce the size of the finally realized surface feature. Nonetheless, large movements of a particular engagement member results in a larger area of vertebral bone that is affected by the engagement member. For example, the amplitude of the movement of the engagement member 168 in FIG. 14 is significantly less than ½ of the length of the footprint of the engagement member 168 on the endplate 162. Thus, when moved between the positions of 168′ and 168″ of FIG. 14, an area in the bone corresponding to the area “D” of FIG. 15 is either scraped away or compacted leaving a surface feature in which the engagement member 168 settles when the movement of the artificial disc 160 is stilled.

The area of vertebral bone affected by the movement of the engagement member 168 in FIG. 14 is substantially less than the area of vertebral bone affected by the movement of the engagement member 168 in FIG. 12. Thus, the smaller amplitude of movement depicted in FIG. 14 provides a lesser amount of disturbance to the adjacent vertebra 200 or 202 along the axis of movement compared to the larger amplitude of movement depicted in FIG. 14. In the embodiment of FIG. 1, the amplitude of movement may be controlled by threading the agitator component 132 further into the body housing 102 for larger amplitudes or further out of the body housing 102 for smaller amplitudes. Alternatively, the amplitude may be a function of electrical power input to the transducer 136.

In alternative embodiments, more complex agitation patterns are employed. By way of example, in one embodiment the amplitude of movement is varied from a larger amplitude when the engagement member is near the surface of the adjacent vertebrae to a smaller amplitude as the engagement member is further embedded into the vertebrae. In a further embodiment, an engagement member is moved in a pattern that includes a cross-axial component as well as the above described axial component, thus affecting an area of bone that is larger than the engagement member in two different axes.

In a further embodiment, the engagement member is moved in a pattern that includes a perpendicular movement component which is aligned with the longitudinal axis of the spine as indicated by the arrows 208 and 210 in FIG. 11. The perpendicular component may be in place of or in addition to the foregoing patterns of movement. Additionally, the perpendicular movement component in a pattern may be simultaneous with an axial component or components or sequential to an axial component or components. Perpendicular movement may be provided by a reciprocating rotary movement of the drive shaft 122 with some modification of the outer sleeve 106. Further, the perpendicular component may be provided by the use of linkages or impact wedges near the tapered end portion 112.

Once the artificial disc 160 has been embedded into the adjacent vertebrae 200 and 202 to the desired depth, the agitator component 132 is deenergized thereby stilling the movement of the artificial disc 160. As the movement of the artificial disc 160 is stilled, the engagement members 169 settle into the respective surface features generated on the adjacent vertebra 100 o 202.

Next, the gripper 142 is disengaged. With reference to FIGS. 4-8, the operator applies a force to the trigger mechanism 128 in the direction of the arrow 184 of FIG. 4. As the force applied to the trigger mechanism 128 increases above the force provided by the internal compression spring 118, the trigger mechanism 128 pivots about the pivot pin 130 forcing the spring retaining screw 120 in the direction of the arrow 186. As the spring retaining screw 120 moves in the direction of the arrow 186, the internal compression spring 118 is compressed and the inner shaft 108 is forced in the direction of the arrow 186. Thus, the throat portion 146 of the gripper 142 is moved in a direction out of the outer sleeve 106 from the position shown in FIG. 8 to the position shown in FIG. 6.

As the throat portion 146 moves out of the outer sleeve 106, the finger pairs 152 and 154 are less constricted by the tapered end portion 112 of the insertion instrument 100. Accordingly, the finger pairs 154 and 156 are resiliently forced in a direction away from the eight notches, 172, 174, 176, 178, 180 and the three notches not shown and the finger pairs 154 and 156 are resiliently forced away from the ledge 182. The artificial disc 160 is thus only firmly gripped by the gripper 142. Accordingly, by forcing the insertion instrument 100 away from the vertebrae 200 and 202, the finger pairs 154 and 156 are forced apart as the slit 150 widens. As the finger pairs 154 and 156 are moved out of and away from the eight notches, 172, 174, 176, 178, 180 and the three notches not shown, the gripper 142 is disengaged from the artificial disc 160. As the gripper 142 clears the artificial disc 160, the gripper returns to its non-stressed condition with the slits 150 and 152 narrowing to the unstressed condition shown in FIG. 2 and the artificial disc 160 remains embedded in the vertebrae 200 and 202 as shown in FIG. 16.

While the present invention has been illustrated by the description of exemplary processes and system components, and while the various processes and components have been described in considerable detail, applicant does not intend to restrict or in any way limit the scope of the appended claims to such detail. Additional advantages and modifications will also readily appear to those ordinarily skilled in the art. By way of example, the gripper and inner shaft of an insertion instrument may be integrally formed. The invention in its broadest aspects is therefore not limited to the specific details, implementations, or illustrative examples shown and described. Accordingly, departures may be made from such details without departing from the spirit or scope of applicant's general inventive concept.

We claim:

1. A method of engaging a bone with an implant comprising:
placing a first surface of an implant adjacent to a first bone portion;
contacting the first bone portion with at least one first engagement member extending from the first surface;
controlling an agitator to agitate the first surface of the implant and the at least one first engagement member;
generating at least one first surface feature in the first bone portion with the agitated at least one first engagement member;
stilling the first surface implant and the at least one first engagement member; and
settling the stilled at least one first engagement member into engagement with the at least one first surface feature.

2. The method of claim 1, further comprising:
placing a second surface of the implant adjacent to a second bone portion;
contacting the second bone portion with at least one second engagement member extending from the second surface;
controlling the agitator to agitate the second surface of the implant and the at least one second engagement member;
generating at least one second surface feature in the second bone portion with the agitated at least one second engagement member;
stilling the second surface implant and the at least one second engagement member; and
settling the stilled at least one second engagement member into engagement with the at least one second surface feature.

3. The method of claim 2, wherein:
placing a first surface of the implant adjacent to a first bone portion comprises placing the first surface of the implant adjacent to a lower side of a first vertebra; and
placing a second surface of the implant adjacent to a second bone portion comprises placing the second surface of the implant adjacent to an upper side of a second vertebra.

4. The method of claim 3, further comprising:
mating a core portion of the implant with a first endplate; and
mating the core portion of the implant with a second endplate, wherein the first endplate comprises the first surface and the second end plate comprises the second surface.

5. The method of claim 2, wherein contacting the first bone portion with at least one first engagement member extending from the first surface comprises;
reducing a distraction force acting on the first bone portion and the second bone portion.

6. The method of claim 5, wherein
agitating the first surface of the implant, agitating the second surface of the implant and reducing the distraction force are performed at least in part simultaneously.

7. The method of claim 1, wherein:
the first surface lies generally in a plane perpendicular to a longitudinal axis of the first bone portion; and
agitating the first surface of the implant comprises moving the first surface generally within the plane.

8. The method of claim 7, wherein agitating the first surface of the implant comprises:
moving the first surface back and forth generally along a single axis within the plane.

9. The method of claim 7, wherein agitating the first surface of the implant comprises:
moving the first surface in a recurring non-linear pattern within the plane.

10. The method of claim 1, wherein:
the first surface lies generally in a plane perpendicular to a longitudinal axis of the first bone portion;
the agitator has an axis that lies within a plane generally parallel to the plane of the first surface; and
agitating the first surface of the implant comprises agitating the first surface in a pattern having a movement component along the longitudinal axis of the first bone portion.

11. The method of claim 10, wherein agitating the first surface of the implant comprises agitating the first surface in a pattern having a movement component along the plane of the first bone portion.

12. The method of claim 1, wherein:
the at least one first engagement member defines a footprint on the first surface with a length along a first axis;
agitating the first surface comprises agitating the first surface to generate at least one movement vector of the first surface parallel to the first axis; and
the at least one movement vector of the first surface parallel to the first axis is less than ½ of the length of the footprint along the first axis.

13. The method of claim 12, wherein:
the at least one first engagement member defines a footprint on the first surface with a length along a second axis, the second axis generally perpendicular to the first axis;
agitating the first surface comprises agitating the first surface to generate a movement vector of the first surface parallel to the second axis; and
the movement vector of the first surface parallel to the second axis is less than ½ of the length of the footprint along the second axis.

14. The method of claim 12, wherein agitating the first surface comprises:
agitating the first surface to generate a first movement vector of the first surface parallel to the first axis; and
agitating the first surface to generate a second movement vector of the first surface parallel to the first axis after the generation of the first movement vector, wherein the second movement vector is shorter than the first movement vector.

15. An implant positioning tool comprising:
a housing;
an agitator located within the housing for providing a recurring pattern of movement; and
a shaft extending out of the housing and having a first end portion operably connected to the agitator and a second end portion configured to operably couple with an implant such that the recurring pattern of movement of the agitator causes the implant to move in a recurring pattern corresponding to the recurring pattern of movement of the agitator.

16. The tool of claim 15, wherein the agitator comprises a transducer for generating a recurring reciprocating pattern.

17. The tool of claim 15, wherein the second end portion comprises:
a gripper configured to couple with an artificial disc, the gripper moveable between a first position wherein the artificial disc is snugly coupled with the artificial disc so as to allow the grippe to couple with and decouple from the artificial disc, and a second position wherein the artificial disc is securely coupled with the artificial disc so as to impede decoupling from the artificial disc.

18. The tool of claim 17, further comprising:
a sleeve extending from the housing and containing at least a portion of the shaft, the sleeve configured to allow reciprocating motion of the at least a portion of the shaft contained therein; and
a tapered end portion located at one end portion of the sleeve, the tapered end portion configured to allow the gripper to couple with and decouple from the artificial disc when the gripper is in the first position, and to force the gripper to impede decoupling from the artificial disc when the gripper is in the second position.
